(19) United States
(12) Patent Application Publication
(10) Pub. No.: US 2005/0069593 A1
(43) Pub. Date: Mar. 31, 2005

(54) Nutritional supplement containing 7-Keto-DHEA and conjugated linoleic acid

(75) Inventor: Jeff Zwiefel, Victoria, MN (US)

Correspondence Address: FAEGRE & BENSON LLP;PATENT DOCKETING, 2200 WELLS FARGO CENTER, MINNEAPOLIS, MN, 55402, US

(73) Assignee: LIFE TIME FITNESS, Inc., Eden Prairie, MN (US)

(21) Appl. No.: 10/952,992

(22) Filed: Sep. 29, 2004

Related U.S. Application Data
(60) Provisional application No. 60/506,787, filed on Sep. 29, 2003.

(51) Int. Cl. A61K 35/78; A61K 33/24; A61K 31/555; A61K 31/57; A61K 31/202

(52) U.S. Cl. 424/655; 514/171; 514/560; 514/184; 424/729

(57) ABSTRACT

The present invention provides a dietary or nutritional supplement for healthy humans that is a combination of 7-oxo-dehydroepiandrosterone (commonly known as “7-oxo-DHEA” or “7-Keto-DHEA”) and conjugated linoleic acid (“CLA”), or nutraceutically acceptable derivatives of these two compounds. In one embodiment, the nutraceutically acceptable derivative of 7-oxo-dehydroepiandrosterone is 3-acetyl-7-oxo-dehydroepiandrosterone. This invention also includes a regimen for supplementing a healthy human's diet by administering the supplement to the human. The invention further includes a regimen for supplementing a healthy human's diet by administering daily to the human 7-oxo-dehydroepiandrosterone and conjugated linoleic acid, or nutraceutically acceptable derivatives of these two compounds.

### REFERENCE TO RELATED APPLICATION

This application claims the benefit of U.S. Provisional Application No. 60/506,787 filed Sep. 29, 2003 and entitled “Nutritional Supplement Containing 7-Keto-DHEA and Conjugated Linoleic Acid,” the disclosure of which is incorporated herein by reference in its entirety.

### TECHNICAL FIELD

Dietary or nutritional supplements are typically designed to compensate for insufficient or reduced levels of nutrients in the modern human diet. One particular goal in supplementing a diet is to increase or enhance the function of tissues such as muscle tissue. For example, in the sporting and athletic community, food supplements which specifically improve athletic ability are increasingly important, such as supplements that promote or enhance physical growth or endurance, or reduce recovery time after exertion. Another goal may be to enhance the rate of fat metabolism in the body, or to inhibit fat storage.

Dehydroepiandrosterone (DHEA) is a hormone linked to healthy aging, including healthy immune brain and cardiovascular functions. DHEA is produced by the adrenal cortex along with other steroids such as glucocorticoids (cortisol or hydrocortisone) and mineral corticoids (aldosterone). DHEA and its metabolite dehydroepiandrosterone-3-sulfate, or “DHEA-S,” are the major secretory steroidal products of the adrenal gland. DHEA is believed to have great importance in human physiology. However, to date, its exact role is unknown.

Concerns have been expressed about the use of DHEA as a dietary supplement, because when it is ingested it is converted into various metabolites, some of which may be convertible into active male or female sex hormones, specifically, testosterone and estrogens.

7-oxo-dehydroepiandrosterone (3-β-hydroxyandrost-5-en-7,17-dione), which is popularly known as “7-oxo-DHEA” or “7-keto-DHEA,” is a metabolite of DHEA and is a natural substance produced in the adrenal gland, gonads and brain. 7-Keto-DHEA has been shown to be effective in enhancing weight loss when combined with a diet and exercise program.

7-keto-DHEA is not only a potent metabolite of DHEA but, unlike DHEA, is not converted in vivo to active androgens and estrogens. Scientific research on 7-keto-DHEA demonstrates that it may have therapeutic applications in immune modulation, immune enhancement through T-cell upregulation, memory enhancement, and weight loss and management. For further information on 7-keto-DHEA, reference may be made to U.S. Pat. No. 5,807,848 to Lardy.

U.S. Pat. No. 5,296,481 to Partridge, et al. reports methods for preventatively treating weight gain and for treating obesity comprising administering a therapeutically effective amount of 7-keto-DHEA. U.S. Pat. No. 5,807,848 to Lardy reports methods for averting weight gain, promoting weight loss, or treating obesity comprising administering an effective amount of an esterified 7-keto-DHEA, 3-acetyl-7-oxo-dehydroepiandrosterone.

Conjugated linoleic acid, popularly known as “CLA,” includes a group of positional and geometric isomers derived from linoleic acid (octadecadienoic acid) and having a conjugated double bond system. CLA is found in some foods, most predominantly in red meats deriving from ruminant mammals, and to a lesser degree in dairy products.

Research has demonstrated that CLA is an in vitro antioxidant, and in cells it protects membranes from oxidative attack. CLA is also reported to have anticarcinogenic properties, antidiabetic properties, cholesterol-lowering effects, and other biological activities in mammalian models. For a review of CLA and its mechanisms of biological action, see Pariza, et al., Proc. Soc. Exp. Biol. Med. 223, 8 (2000), Pariza, et al., Prog. Lipid Res. 40, 283 (2001), and Yurawecz, et al. (eds.), Advances in Conjugated Linoleic Acid Research (AOCS Press, 1999).

U.S. Pat. Nos. 5,554,646, and 5,430,066 to Cook, et al. report methods for reducing body fat, preventing weight gain, or preserving or increasing body protein content in an animal by administering an effective amount of CLA.

### SUMMARY OF THE INVENTION

The present invention provides a nutritional supplement for a healthy human that is a combination of 7-oxo-dehydroepiandrosterone and conjugated linoleic acid, or nutraceutically acceptable derivatives of these two compounds. In one embodiment, the nutraceutically acceptable derivative of 7-oxo-dehydroepiandrosterone is 3-acetyl-7-oxo-dehydroepiandrosterone. This invention also includes a regimen for supplementing a healthy human's diet by administering the supplement to the human. The invention further includes a regimen for supplementing a healthy human's diet by administering daily to the human 7-oxo-dehydroepiandrosterone and conjugated linoleic acid, or nutraceutically acceptable derivatives of these two compounds.

### DETAILED DESCRIPTION OF THE INVENTION

Nutritional Supplements

In one embodiment, the present invention provides a dietary or nutritional supplement for a healthy human that is a combination of 7-oxo-dehydroepiandrosterone and conjugated linoleic acid, or nutraceutically acceptable derivatives of these two compounds.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease.

Since conjugated linoleic acid is naturally occurring in, and can be extracted from, digestible foodstuff, the label “nutraceutical” may be applied to it. As discussed above, 7-keto-DHEA is naturally produced in the adrenal gland, gonads and brain, and has been demonstrated to have physiological benefits. Thus, for the sake of simplicity, the term “nutraceutical” will also be applied to 7-keto-DHEA herein, and to the supplements described herein.

As used herein, the phrase “nutraceutically acceptable derivative” is used to refer to a derivative or substitute for the stated chemical species that operates in a similar manner to produce the intended effect, and is structurally similar and physiologically compatible. In the following discussion of the invention, it should be understood that the term “CLA” or “conjugated linoleic acid,” and “7-oxo-dehydroepiandrosterone” or “7-keto-DHEA” can refer to either the stated chemical species or to a nutraceutically acceptable derivative thereof.

By way of example only, such substitutes could include salts, esters, hydrates, or complexes of the stated chemical. Such substitutes could also include stereoisomers or structural isomers, so long as the substitute operates similarly and produces the desired effect. Alternatively, the substitute could be a precursor to the stated chemical, which subsequently undergoes a reaction in vivo to yield the stated chemical or a substitute thereof.

7-Keto-DHEA

The supplements of the present invention comprise 7-oxo-dehydroepiandrosterone, or a nutraceutically acceptable derivative. 7-Oxo-dehydroepiandrosterone, which is popularly known as 7-oxo-DHEA or 7-keto-DHEA, is a metabolite of DHEA and is a natural substance produced in the adrenal gland, gonads and brain. The chemical structure of 7-keto-DHEA is given in FIG. 1. For further information on 7-keto-DHEA, reference may be made to U.S. Pat. No. 5,296,481 to Partridge, et al. and U.S. Pat. Nos. 5,292,730, 5,585,371, 5,641,766, and 5,807,848 to Lardy.

[image]

Nutraceutically acceptable derivatives of 7-keto-DHEA may include compounds derivatized to include a hydroxy group at the 7 position in place of the carbonyl oxygen. Likewise, suitable derivatives may include compounds derivatized to include a hydroxy group at the 17 position in place of the keto oxygen. Suitable derivatives of 7-keto-DHEA may also include compounds derivatized at the 3 position to yield a salt, a sulfate ester, or an ester such as an acetate. Other suitable derivatives are described in U.S. Pat. No. 5,296,481 to Partridge, et al. and U.S. Pat. Nos. 5,292,730, 5,585,371, 5,641,766, and 5,807,848 to Lardy. It is desirable, though not required, that the nutraceutically acceptable derivative is not converted in vivo to DHEA, since DHEA can subsequently be converted into estrogenic or androgenic hormones.

A particularly suitable derivative for the practice of the present invention is 3-acetyl-7-oxo-dehydroepiandrosterone (3-β-acetoxyandrost-5-en-7,17-dione), which may be called “3-acetyl-7-keto-DHEA,” “7-oxo-DHEA acetate,” or “7-keto-DHEA acetate,” but is sometimes improperly referred to as “7-keto-DHEA.” The structure of 3-acetyl-7-keto-DHEA is given in FIG. 2. 3-Acetyl-7-keto-DHEA is reported to be metabolized in vivo to 7-keto-DHEA, which is in turn converted to an active sulfate ester.

[image]

U.S. Pat. No. 5,296,481 to Partridge, et al. and U.S. Pat. Nos. 5,292,730, 5,585,371, 5,641,766, and 5,807,848 to Lardy report methods for synthesizing 7-keto-DHEA and 3-acetyl-7-keto-DHEA. 3-Acetyl-7-keto-DHEA is commercially available from Humanetics Corporation (Minneapolis, Minn.).

Conjugated Linoleic Acid

The supplements of the present invention also comprise conjugated linoleic acid, or a nutraceutically acceptable derivative. Conjugated linoleic acid (popularly known as “CLA”) includes a group of positional and geometric isomers derived from linoleic acid (octadecadienoic acid) and having a conjugated double bond system. Linoleic acid is a fatty acid that includes a chain of eighteen carbon atoms and two carbon-carbon double bonds. Generally the double bonds are separated by a saturated methylene group, and therefore the double bonds are not conjugated. In linoleic acid found in forages and seed oils, the double bonds connect carbons 9 and 10 and carbons 12 and 13. Each double bond has a cis configuration.

CLA comprises two conjugated carbon-carbon double bonds in the eighteen-carbon chain, with the first double bond position most often connecting carbons 9 and 10, or carbons 10 and 11. Each double bond can take either the cis or trans configuration. The cis-9, trans-11 and trans-10, cis-12 isomers are the predominant isomers found in foods, although trans-7, cis-9 is found in fairly significant quantities in milk fat. The chemical structures of cis-9, trans-11 CLA and trans-10, cis-12 CLA are given in FIG. 3.

[image]

It is thought that any nutraceutically acceptable derivative of CLA would be required to maintain conjugation with at least two carbon-carbon double bonds, and to be derivatized at the acid terminus in such a manner that the acid group could be regenerated in vivo. Nutraceutically acceptable derivatives of CLA may therefore include salts or esters. By way of example, U.S. Pat. No. 6,432,453 to Krumhar reports the reaction of CLA with glycerol to form an ester, resulting in a stabilized form of CLA.

Suitable derivatives of CLA may include additional sites of unsaturation, whether conjugated into the diene system or isolated from the conjugated diene. Suitable derivatives may also include branching sites in the alkyl chain, or short alkyl substituents (one to three carbons) appended to the alkyl chain.

As used herein with respect to the supplements of the invention, the phrase “conjugated linoleic acid” can refer to any isomer of CLA, or to a mixture of two or more isomers of CLA. In general, the supplements of the invention will include a mixture of isomers.

Particular isomers of CLA may be more suitable or more effective in the supplements of the present invention than others. The cis-9, trans-11 and trans-10, cis-12 isomers are reported to be the predominate isomers found in foods. The cis-9, trans-11 and trans-9, cis-11 isomers may be the isomers most responsible for physiological activity or effect, while trans-10, cis-12 may increase fatty acid oxidation; see Evans, et al., J. Nutr. 132, 450 (2002).

CLA is commercially available as a mixture of isomers under the trade name TONALIN from Natural, Inc. (Vernon Hills, Ill.). TONALIN CLA is produced using a proprietary process that converts linoleic acid in natural safflower oil into conjugated linoleic acid. CLA is also commercially available from Loders Croklaan (Wormerveer, Holland), and is commercially available as a raw material from Conlinco, Inc. (Detroit Lakes, Minn.). A suitable product containing a calcium salt of is available from FutureCeuticals (Momence, Ill.). Supplements containing CLA are widely available from other manufacturers or distributors.

CLA is found in some foods, most predominantly in red meats deriving from ruminant mammals, and to a lesser degree in dairy products. Therefore, CLA could be obtained by extraction from food sources. However, synthesis of CLA from extractable food oils is more efficient. Methods for synthesizing CLA are reported in U.S. Pat. No. 6,524,527 to Fimreite, et al., U.S. Pat. No. 6,420,577 to Reaney, et al., U.S. Pat. No. 6,410,761 to Saebo, et al., U.S. Pat. No. 6,015,833 to Saebo, et al., U.S. Pat. No. 5,986,116 to Iwata, et al.,

Methods for obtaining compositions enriched in a particular isomer of CLA are described, for example, in U.S. Pat. Nos. 6,465,666, 6,242,621 and 6,060,514 to Jerome, et al., U.S. Pat. No. 6,380,409 to Saebo, et al., and U.S. Pat. Nos. 6,333,353 and 6,225,486 to Saebo, et al.

Composition and Use of the Nutritional Supplement

In one embodiment, the present invention provides a thermogenic supplement comprising a combination of 7-oxo-dehydroepiandrosterone and conjugated linoleic acid, or nutraceutically acceptable derivatives of these two compounds, and optional additives such as green tea extract, trivalent chromium, or ginseng, or a combination of any or all of the foregoing. The term “thermogenic” means that the supplement induces fat loss characterized by an elevated core body temperature or by an increased caloric use as a result of a heightened metabolic state.

In one embodiment the supplement may comprise, per gram of supplement, about 5 milligrams to about 30 milligrams of 7-oxo-dehydroepiandrosterone, a nutraceutically acceptable derivative, or a combination thereof. In another embodiment, the supplement comprises about 5 milligrams to about 30 milligrams of 3-acetyl-7-oxo-dehydroepiandrosterone per gram of supplement. In yet another embodiment, the supplement comprises about 10 milligrams to about 20 milligrams 3-acetyl-7-oxo-dehydroepiandrosterone per gram of supplement.

In one embodiment the supplement may comprise, per gram of supplement, about 0.5 gram to about 0.9 gram of conjugated linoleic acid, a nutraceutically acceptable derivative, or a combination thereof. In another embodiment, the supplement comprises about 0.6 gram to about 0.8 gram conjugated linoleic acid per gram of supplement. In yet another embodiment, cis-9, trans-11 and trans-10, cis-12 isomers are the predominant conjugated linoleic acid isomers in the supplement.

One embodiment of the supplement comprises, per gram of supplement, about 10 to about 20 milligrams 3-acetyl-7-oxo-dehydroepiandrosterone and about 0.6 to about 0.8 gram conjugated linoleic acid.

The supplement may optionally include inactive ingredients such as diluents or fillers, viscosity-modifying agents, preservatives, flavorings, colorants, or other additives conventional in the art. By way of example only, conventional ingredients such as beeswax, lecithin, gelatin, glycerin, caramel, and carmine may be included.

In place of or in combination with CLA, the nutritional supplement of the present invention may comprise L-camitine, hydroxycitric acid, or garcinia cambogia. The ingredient may take the form of a tincture, a dry extract, a powder, or other suitable form as is known in the art.

The supplement may also include any or all of the optional ingredients described below, which are expected to contribute to the thermogenic properties of the supplement. Other ingredients known to promote thermogenesis may also be suitable for use in the supplement. It is preferred that the nutritional supplement does not contain ephedra, due to known adverse health effects that have been linked to the use of ephedra as a nutritional supplement.

The supplement may suitably be presented in the form of a softgel. A softgel may be made using conventional methods. In general, a softgel includes a layer of gelatin encapsulating a small quantity of the supplement. The total mass of a single softgel is on the order of one gram. The supplement may also be suitably presented as a liquid-filled and sealed gelatin capsule, which may be made using conventional methods. The supplement may suitably be presented in other forms including as a dispensable liquid, for example.

The supplement of the present invention is preferably taken daily. However, consumption at lower frequency, such as several times per week or even isolated doses, may also have some benefit.

The supplement of the present invention should preferably be ingested with or after a meal. An upset stomach or other side effect may be experienced if the supplement is taken on an empty stomach. In particular, the supplement may be most effective if taken at the time of a person's morning meal, or at the time of a person's noontime meal.

For daily consumption, it may be advantageous to consume a portion of the supplement at the time of the human's morning meal, and a second portion of the supplement at the time of the human's noontime meal. In this embodiment, a portion may be administered shortly before, during, or shortly after the meal. Generally, the morning portion and the noontime portion will each provide approximately the same quantity of 7-oxo-dehydroepiandrosterone and conjugated linoleic acid. Alternatively, the supplement may be taken at the time of the morning meal, and again at the time of the evening meal.

The supplement and regimens of the present invention are most effective when combined with a balanced diet according to generally accepted nutritional guidelines, and a program of modest to moderate exercise several times a week.

Optional Ingredients

Green Tea Extract

Green tea extract contains naturally occurring antioxidants, and ingestion of green tea extract is thought to increase energy, burn fat, and protect against disease.

The composition of tea is determined by soil conditions, the season the leaves are harvested, and the method used to process the tea. As a rough guide, green tea contains 2.9 to 4.2% caffeine, 0.02 to 0.04% theophylline, and 0.15 to 0.2% theobromine. Green tea also contains a number of polyphenolic compounds. The catechin epigallocatechin gallate (EGCG) is the most abundant (about 50% of total tea catechins), and is thought to be one of the most pharmacologically active. The other main catechins are epicatechin (EC), epicatechin gallate (ECG), and epigallocatechin (EGC).

The color (green, oolong, and black) of tea (camellia sinensis) is determined by how the tea leaves are processed. Green tea is prepared in such a way as to avoid the oxidation of polyphenols in the tea leaves.

Scientists have found that green tea stimulates thermogenesis and this effect cannot be completely attributed to its caffeine content because the thermogenic effect of green tea is greater than an equivalent amount of caffeine. An in vitro study by Dulloo, et al. [Int. J. Obes. Relat. Metab. Disord. 24, 252 (2000)] found that a catechin that is abundant in green tea, EGCG, increased the respiration rate of brown adipose tissue. The study concluded that EGCG was effective to stimulate thermogenesis.

In addition to its thermogenic properties, green tea has a variety of well-documented health benefits. Green tea is an antioxidant that has a strong anticancer effects in skin, stomach, colon, etc.

Green tea extract is generally obtained by an organic-solvent extraction or a water/solvent extraction from dried tea leaves. By way of example, ethyl acetate is suitable as a solvent for extraction; alternatively, a water/alcohol mixture is suitable for extraction. Green tea extract is commercially available from a variety of sources, including FutureCeuticals (Momence, Ill.) and TCT Fine Chemical Inc. (Fairfield, N.J.). One suitable green tea extract contains about 25% caffeine and about 30-40% EGCG. Another suitable green tea extract contains about 98% polyphenols and about 45% EGCG.

In one embodiment, the supplement comprises about 40 to about 200 milligrams green tea extract per gram of supplement.

Caffeine

In place of or in combination with green tea extract, the nutritional supplement of the present invention may comprise caffeine. In one embodiment, the supplement further comprises about 2 to about 50 milligrams caffeine per gram of supplement.

Other suitable substitutes for Green Tea Extract

In place of or in combination with green tea extract, the nutritional supplement of the present invention may comprise cayenne or black pepper, ginger root, betaine, gotu kola, or gurana. The ingredient may take the form of a tincture, a dry extract, a powder, or other suitable form as is known in the art.

Trivalent Chromium

Chromium is an essential nutrient that helps regulate metabolism, carbohydrate attention in recent years. Chromium deficiency in humans has been reported as a cause of a reduced response of insulin-sensitive tissue to insulin, manifested by impaired glucose metabolism. The action of chromium is closely associated with mechanisms involving insulin. Chromium possesses properties which both mimic and enhance the effects of insulin. Chromium enhances the effects of insulin by indirectly assisting amino acid uptake by muscles, stimulates protein synthesis and retards the rate of protein breakdown.

In particular, trivalent chromium compounds are safe and required in a proper diet of animals and humans. In contrast, hexavalent chromium compounds are toxic and should not be ingested. As used throughout the remainder of this Specification, the term “chromium” refers to trivalent chromium, or to a complex comprising trivalent chromium chelated to a physiologically acceptable chelating agent.

Many clinical studies with supplemental chromium have shown only modest benefit due to poor absorption of nutritional (trivalent) chromium. Adequate absorption of chromium generally occurs only when the metal is associated with a natural chelating agent, such as picolinic acid.

For the supplement of the present invention, it is preferred that chromium be provided in a physiologically safe and biologically active form, particularly as chromium picolinate or chromium polynicotinate, for example. Chromium picolinate is described in U.S. Pat. No. 4,315,927 to Evans. Chromium polynicotinate, which is niacin-bound chromium (III), is described in U.S. Pat. No. 5,194,615 to Jensen. The phrases “chromium nicotinate” and “chromium polynicotinate” should be understood to be interchangeable.

Other suitable forms of trivalent chromium include “glucose tolerance factor” (GTF) complex, which is described in U.S. Pat. No. 5,194,615 to Jensen, and chromium (III) chloride (CrCl₃), which may optionally be hydrated.

While the supplement may include only one form of chromium, it is preferred that the supplement include a mixture of chromium compounds. One suitable mixture comprises chromium picolinate and chromium polynicotinate. By way of example, the picolinate and polynicotinate compounds may be mixed in an appropriate ratio to provide an equal number of moles of trivalent chromium.

In one embodiment, the supplement further comprises about 50 to about 1000 micrograms of a trivalent chromium compound per gram of supplement.

Bitter Orange Extract

The unripe peel of bitter orange (citrus aurantium) contains appreciable quantities of neohesperidin (about 14%), and adrenergic amines including synephrine, N-methyltyramine, hordenine, octopamine, and tyramine.

It is believed that ingestion of a combination of the above-listed adrenergic amines can stimulate beta-3 receptors with minimal impact on other receptor sites. Stimulation of beta-3 receptors functions to increase metabolic rate without affecting heart rate or blood pressure. Stimulation of the beta-3 receptor sites elicits lipolysis (the breakdown of fat) and increases the metabolic rate (thermogenesis). Synephrine is a well-known bronchial dilator, and is used extensively in diet pills and weight loss formulas. It is commonly used as a substitute for ephedrine and caffeine. Synephrine offers all the advantages of a stimulant, without causing the same negative effects.

Bitter orange extract is commonly available with a synephrine content of about 1.5% to about 6% or greater. Bitter orange extract is commercially available in a variety of synephrine potencies from Northwest Botanicals, Inc. (Grant's Pass, Oreg.) and TCT Fine Chemical Inc. (Fairfield, N.J.). By way of example, a bitter orange extract standardized to about 6% synephrine content is suitable for the compositions of the present invention.

In one embodiment, the supplement comprises about 20 to about 100 milligrams bitter orange extract per gram of supplement.

Synephrine

In place of or in combination with bitter orange extract, the nutritional supplement of the present invention may comprise synephrine. Synephrine is a well-known bronchial dilator, and is used extensively in diet pills and weight loss formulas. It is commonly used as a substitute for ephedrine and caffeine. Synephrine offers all the advantages of a stimulant, without causing the same negative effects.

In one embodiment, the supplement further comprises about 1 to about 25 milligrams

Other Suitable Substitutes for Bitter Orange Extract

In place of or in combination with bitter orange extract, the nutritional supplement of the present invention may comprise ephedra or ma huang, pyruvate, octopamine, forsoklii, caffeine, or chitosan. The ingredient may take the form of a tincture, a dry extract, a powder, or other suitable form as is known in the art.

Ginseng

Ginseng is a general stimulant, and is considered to be an adaptogen (i.e., it can increase the body's ability to tolerate stressful situations). It is commonly taken to increase energy and to enhance physical stamina. Ginseng grows as a perennial plant native to regions of China, North Korea, and Siberia. The root of the plant is dug in the fall, washed, steamed, and dried for use in herbal medicine.

There are numerous triterpenoid saponins, referred to as “ginsenosides,” in the different species of ginseng plants. These ginsenosides are thought to be the most important active components of the plant extract. The ginsenosides are believed to have a stimulatory effect on the immune system and fight fatigue and stress by supporting the adrenal glands and by increasing the use of oxygen by the muscles.

Many other components, thought to be minor, have also been isolated from ginseng. In addition, ginseng contains vitamin A, vitamin E, and B-complex vitamins including thiamin, riboflavin, B-12, and niacin. Ginseng also contains minerals such as calcium, iron, phosphorus, sodium, silicon, potassium, manganese, magnesium, and sulfur.

Two types of ginseng are commonly available: Asian (or “Korean”) ginseng (Panax ginseng), and American ginseng (Panax quinquefolium). American Ginseng contains a higher percentage of ginsenosides than Asian ginseng. A third plant, Eleutherococcus senticosus, is actually a different species but is often referred to as “Siberian ginseng.” Each of these forms of ginseng is suitable for use in the present invention.

For use in the supplement of the present invention, ginseng may take the form of a tincture, a dry extract, a powder, or other suitable form as is known in the art.

In one embodiment, the supplement comprises about 20 to about 250 milligrams ginseng root per gram of supplement. In another embodiment, the supplement includes a mixture of ginsengs, such as a mixture of American ginseng root and Siberian ginseng root. By way of example only, the supplement may include a mixture of about 20 to about 200 milligrams American ginseng root and about 5 to about 50 milligrams Siberian ginseng root per gram of supplement.

Regimen for Diet Supplementation

In another embodiment, the invention provides a regimen for supplementing a human's diet, comprising administering to the human a supplement comprising a combination of 7-oxo-dehydroepiandrosterone and conjugated linoleic acid, or nutraceutically acceptable derivatives of these two compounds.

In yet another embodiment, the invention provides a regimen for supplementing a human's diet, comprising administering to the human about 25 to about 200 milligrams 7-oxo-dehydroepiandrosterone and about 0.5 to about 5 grams conjugated linoleic acid, or nutraceutically acceptable derivatives of these two compounds, on a daily basis.

In one embodiment of the regimen, about 25 to about 200 milligrams of 3-acetyl-7-oxo-dehydroepiandrosterone is administered to the human on a daily basis. In another embodiment, about 50 milligrams of 3-acetyl-7-oxo-dehydroepiandrosterone is administered to the human on a daily basis.

In yet another embodiment, about 1.5 grams to about 3 grams of conjugated linoleic acid is administered to the human on a daily basis. About 2 grams of conjugated linoleic acid is administered to the human on a daily basis in another embodiment of the regimen.

Alternatively, appropriate dosages may be determined based on the human's body mass. In one embodiment, about 0.25 milligram to about 2 milligram 3-acetyl-7-oxo-dehydroepiandrosterone per kilogram of the human's body weight is administered to the human on a daily basis. In another embodiment, about 0.5 milligram to about 1 milligram 3-acetyl-7-oxo-dehydroepiandrosterone per kilogram of the human's body weight is administered to the human on a daily basis.

In another embodiment of the regimen, about 10 milligrams to about 80 milligrams conjugated linoleic acid per kilogram of the human's body weight is administered to the human on a daily basis. In yet another embodiment, about 20 milligrams to about 40 milligrams conjugated linoleic acid per kilogram of the human's body weight is administered to the human on a daily basis.

The supplements described above are suitable for use in the regimens of the present invention. In one embodiment of the regimen, about 3 to about 6 grams of a supplement is administered to the human on a daily basis.

In another embodiment of the regimen, a portion of the supplement is administered at the time of the human's morning meal, and a second portion of the supplement is administered at the time of the human's noontime meal. In this embodiment, a portion may be administered shortly before, during, or shortly after the meal. Generally, the morning portion and the noontime portion will each provide approximately the same quantity of 7-oxo-dehydroepiandrosterone and conjugated linoleic acid.

The regimen may include administering a supplement comprising any or all of the optional ingredients described above. For example, the regimen may comprise administering about 100 to about 400 micrograms of a trivalent chromium compound to the human on a daily basis. In another embodiment, about 200 micrograms of the trivalent chromium compound is administered to the human on a daily basis. The regimen may comprise administering about 40 to about 1000 milligrams green tea extract to the human on a daily basis. In another embodiment, about 400 milligrams green tea extract is administered to the human on a daily basis. The regimen may comprise administering about 50 milligrams to about 500 milligrams ginseng root to the human on a daily basis. In another embodiment, about 300 milligrams American ginseng root and about 20 milligrams Siberian ginseng root is administered to the human on a daily basis.

This invention may take on various modifications and alterations without departing from the spirit and scope thereof. Accordingly, it is to be understood that this invention is not to be limited to the above-described. It is also to be understood that this invention may be suitably practiced in the absence of any element not specifically disclosed herein.

In describing preferred embodiments of the invention, specific terminology is used for the sake of clarity. The invention, however, is not intended to be limited to the specific terms so selected, and it is to be understood that each term so selected includes all technical equivalents that operate similarly.

### EXAMPLE 1

A nutritional supplement according to the present invention was prepared by combining the ingredients listed in Table 1, using conventional methods to yield a composition that was suitable for use as a softgel fill.

TABLE 1
Formulation for nutritional supplement.

| | mg/ | Wt.- % in |
|---|---|---|
| Component | dosage unit | Supplement |
| CLA, 78% | 641 | 74.89 |
| Green Tea Extract (24.5% caffeine) | 100 | 11.68 |
| Chromium Polynicotinate | 0.25 | 0.029 |
| Chromium Picolinate | 0.21 | 0.025 |
| Citrus Aurantium (6% synephrine) | 50 | 5.84 |
| Beeswax | 36 | 4.21 |
| Lecithin | 16 | 1.87 |
| 7-oxo-dehydroepiandrosterone | 12.5 | 1.46 |
| Total | 855.96 | |

Softgel dosage units were then prepared by conventional methods, using the composition as filler. Each softgel dosage unit contained approximately 856 mg of the composition, as detailed in Table 1.

The recommended usage of the softgel dosage units is in a supplementary regimen wherein two dosage units are administered in the morning, and two are administered in the afternoon. Preferably, the regimen is used in conjunction with a program of at least moderate cardiovascular exercise several times per week. More preferably, the regimen is used in conjunction with the aforementioned program cardiovascular exercise and including resistance or weight training several times per week.

### EXAMPLE 2

A nutritional supplement according to the present invention can be prepared by combining the ingredients listed in Table 2, using conventional methods to yield a composition that is suitable for use as a softgel fill.

TABLE 2
Formulation for nutritional supplement.

| | mg/ | Wt.- % in |
|---|---|---|
| Component | dosage unit | Supplement |
| CLA, 78% | 625 | 73.71 |
| Green Tea Extract (45% EGCG, 98% | 100 | 11.79 |
| Polyphenols) | | |
| Green Tea Leaf | 5 | 0.59 |
| Chromium Polynicotinate | 0.25 | 0.029 |
| Chromium Picolinate | 0.21 | 0.025 |
| American Ginseng root | 75 | 8.84 |
| Siberian Ginseng root | 5 | 0.59 |
| Beeswax | 25 | 2.95 |
| 7-oxo-dehydroepiandrosterone | 12.5 | 1.46 |
| Total | 847.96 | |

Softgel dosage units were then prepared by conventional methods, using the composition as filler. Each softgel dosage unit contained approximately 848 mg of the composition, as detailed in Table 2.

The recommended usage of the softgel dosage units is in a supplementary regimen wherein two dosage units are administered in the morning, and two are administered in the afternoon. Preferably, the regimen is used in conjunction with a program of at least moderate cardiovascular exercise several times per week. More preferably, the regimen is used in conjunction with the aforementioned program cardiovascular exercise and including resistance or weight training several times per week.

1. A nutritional supplement for use by a human, comprising, per gram of supplement:
about 5 milligrams to about 30 milligrams of 7-oxo-dehydroepiandrosterone, a nutraceutically acceptable derivative, or a combination thereof; and
about 0.5 gram to about 0.9 gram of conjugated linoleic acid, a nutraceutically acceptable derivative, or a combination thereof.

2. The nutritional supplement of claim 1, comprising about 5 milligrams to about 30 milligrams of 3-acetyl-7-oxo-dehydroepiandrosterone per gram of supplement.

3. The nutritional supplement of claim 1, comprising about 10 milligrams to about 20 milligrams 3-acetyl-7-oxo-dehydroepiandrosterone per gram of supplement.

4. The nutritional supplement of claim 1, comprising about 0.6 gram to about 0.8 gram conjugated linoleic acid per gram of supplement.

5. The nutritional supplement of claim 1, wherein cis-9, trans-11 and trans-10, cis-12 isomers are the predominant conjugated linoleic acid isomers in the supplement.

6. The nutritional supplement of claim 1, comprising, per gram of supplement:
about 10 to about 20 milligrams 3-acetyl-7-oxo-dehydroepiandrosterone; and
about 0.6 to about 0.8 gram conjugated linoleic acid.

7. The nutritional supplement of claim 1, further comprising about 50 micrograms to about 1000 micrograms of a trivalent chromium compound per gram of supplement.

8. The nutritional supplement of claim 7, wherein the trivalent chromium compound is selected from the group consisting of chromium picolinate, chromium polynicotinate, chromium (III) chloride, and mixtures thereof.

9. The nutritional supplement of claim 1, further comprising about 40 milligrams to about 200 milligrams green tea extract per gram of supplement.

10. The nutritional supplement of claim 1, further comprising about 2 milligrams to about 50 milligrams caffeine per gram of supplement.

11. The nutritional supplement of claim 1, further comprising about 20 milligrams to about 250 milligrams ginseng root per gram of supplement.

12. The nutritional supplement of claim 1, further comprising about 20 to about 200 milligrams American ginseng root and about 5 to about 50 milligrams Siberian ginseng root per gram of supplement.

13. The nutritional supplement of claim 1, in the form of a softgel.

14. The nutritional supplement of claim 1, comprising, per gram of supplement:
about 5 milligrams to about 30 milligrams of 3-acetyl-7-oxo-dehydroepiandrosterone;
about 0.5 gram to about 0.9 gram of conjugated linoleic acid;
about 50 to about 1000 micrograms of a trivalent chromium compound selected from the group consisting of chromium picolinate, chromium polynicotinate, chromium (III) chloride, and mixtures thereof;
about 40 milligrams to about 200 milligrams green tea extract; and
about 20 to about 250 milligrams ginseng root.

15. The nutritional supplement of claim 1, comprising, per gram of supplement:
about 10 milligrams to about 20 milligrams of 3-acetyl-7-oxo-dehydroepiandrosterone;
about 0.6 gram to about 0.8 gram of conjugated linoleic acid;
about 50 to about 300 micrograms of a trivalent chromium compound selected from the group consisting of chromium picolinate, chromium polynicotinate, chromium (III) chloride, and mixtures thereof;
about 40 milligrams to about 200 milligrams green tea extract; and
about 20 to about 250 milligrams ginseng root.

16. A regimen for supplementing a human's diet, comprising administering to the human a supplement comprising, per gram of supplement:
about 5 milligram to about 30 milligrams of 7-oxo-dehydroepiandrosterone, a nutraceutically acceptable derivative, or a combination thereof; and
about 0.5 gram to about 0.9 gram of conjugated linoleic acid, a nutraceutically acceptable derivative, or a combination thereof.

17. The regimen of claim 16, wherein a portion of the supplement is administered at the time of the human's morning meal, and a second portion of the supplement is administered at the time of the human's noontime meal.

18. A regimen for supplementing a healthy human's diet, comprising administering to the human, on a daily basis:
about 25 milligrams to about 200 milligrams of 7-oxo-dehydroepiandrosterone, a nutraceutically acceptable derivative, or a combination thereof; and
about 0.5 gram to about 5 grams of conjugated linoleic acid, a nutraceutically acceptable derivative, or a combination thereof.

19. The regimen of claim 18, wherein about 25 to about 200 milligrams of 3-acetyl-7-oxo-dehydroepiandrosterone is administered to the human on a daily basis.

20. The regimen of claim 18, wherein about 50 milligrams of 3-acetyl-7-oxo-dehydroepiandrosterone is administered to the human on a daily basis.

21. The regimen of claim 18, wherein about 1.5 grams to about 3 grams of conjugated linoleic acid is administered to the human on a daily basis.

22. The regimen of claim 18, wherein about 2 grams of conjugated linoleic acid is administered to the human on a daily basis.

23. The regimen of claim 18, further comprising administering about 100 to about 400 micrograms of a trivalent chromium compound to the human on a daily basis.

24. The regimen of claim 18, further comprising administering about 40 to about 1000 milligrams green tea extract to the human on a daily basis.

25. The regimen of claim 18, further comprising administering about 50 milligrams to about 500 milligrams ginseng root to the human on a daily basis.

26. The regimen of claim 18, further comprising administering about 300 milligrams American ginseng root and about 20 milligrams Siberian ginseng root to the human on a daily basis.

27. The regimen of claim 18, wherein a portion containing both 7-oxo-dehydroepiandrosterone and conjugated linoleic acid is administered at the time of the human's morning meal, and a second portion is administered at the time of the human's noontime meal.
